(19) United States
(12) Patent Application Publication
(10) Pub. No.: US 2007/0167419 A1
(43) Pub. Date: Jul. 19, 2007

(54) TREATMENT FOR EMBOLIC STROKE

(75) Inventors: Paul A. Lapchak, San Diego, CA (US); Dalia Araujo, San Diego, CA (US); Justin A. Zivin, Rancho Santa Fe, CA (US)

Correspondence Address: BUCHANAN, INGERSOLL & ROONEY LLP, P.O. BOX 1404, ALEXANDRIA, VA, 22313-1404, US

(73) Assignee: THE REGENTS OF THE UNIVERSITY OF CALIFORNIA, Oakland, CA (US)

(21) Appl. No.: 11/693,125

(22) Filed: Mar. 29, 2007

Related U.S. Application Data
(63) Continuation-in-part of application No. PCT/US06/07412, filed on Mar. 1, 2006.
(60) Provisional application No. 60/658,274, filed on Mar. 2, 2005.
(60) Provisional application No. 60/801,887, filed on May 19, 2006.

(51) Int. Cl. A61K 31/573; A61K 31/445; A61K 31/4015; A61K 31/21; A61K 31/13

(52) U.S. Cl. 514/171; 514/345; 514/424; 514/509; 514/645; 514/327; 514/389

(57) ABSTRACT

Provided herein are methods and compositions for treating stroke that include contacting a subject suffering from a stroke with (1) an antioxidant; (2) an antioxidant and one or more of (i) a thrombolytic agent, (ii) an NMDA receptor antagonist and (iii) a spin trap agent; or (3) a thrombolytic agent in combination with one or more of (i) an NMDA receptor antagonist and (ii) a spin trap agent.

### CROSS REFERENCE TO RELATED APPLICATIONS

This application is a continuation-in-part and claims priority to International Application No. PCT/US2006/007412, filed Mar. 1, 2006, which application claims priority under 35 U.S.C. §119 to U.S. Provisional Application Ser. No. 60/658,274, filed Mar. 2, 2005. This application also claims priority under 35 U.S.C. §119 to U.S. Provisional Application Ser. No. 60/801,887, filed May 19, 2006. The disclosures of all of the above applications are incorporated herein by reference.

### STATEMENT REGARDING FEDERALLY SPONSORED RESEARCH

This invention was supported in part by Grant Nos. NS28121 and NS42244 awarded by the National Institute of Health. The government may have certain rights in this invention.

### BACKGROUND

Acute ischemic stroke is estimated to affect ˜2-2.5 out of every thousand people, resulting upwards of 4.5 million deaths per year worldwide and 9 million stroke survivors, with costs currently exceeding $50 billion in the U.S. alone. Strokes, or cerebrovascular accidents, are the result of an acute obstruction of cerebral blood flow to a region of the brain. There are approximately 500,000 cases each year in the United States, of which 30% are fatal, and hence stroke is the third leading cause of death in the United States. Approximately 80% of strokes are “ischemic” and result from an acute occlusion of a cerebral artery with resultant reduction in blood flow. The remainder are “hemorrhagic”, which are due to rupture of a cerebral artery with hemorrhage into brain tissue and consequent obstruction of blood flow due to lack of flow in the distal region of the ruptured vessel and local tissue compression, creating ischemia.

Stroke commonly affects individuals older than 65 years, and the most powerful risk factor is hypertension. Until recently, there was no approved therapy for acute stroke, which was treated by general medical support only, followed by rehabilitation from the observed damage. In 1996, the FDA approved the use of tissue plasminogen activator (tPA) as therapy for acute ischemic stroke, based on a limited number of controlled trials. Some, but not all, of the trials revealed a 30-55% improvement in clinical outcome, with an overall reduction in morbidity, but not mortality.

### SUMMARY

The invention provides methods and compositions useful for treating ischemic stroke. The invention provides polyphenols and flavonols useful for treating stroke. In addition, the invention provides combination therapies comprising tPA and spin-trap agents for the treatment of ischemic stroke.

The invention shows that natural products, such as Chlorogenic acid, Baicalein and/or Fisetin, when administered after an embolic stroke, can significantly reduce stroke-induced behavioral deficits. The embolic stroke model used in these studies shows that both classes of drug can improve motor function following strokes.

In another embodiment, a method of treating a stroke victim by administering a polyphenol, is provided. Exemplary polyphenols include phenolic acid, or derivative thereof. Exemplary phenolic acids include hydroxycinnimac acid, or derivatives thereof, and hydroxybenzoic acid, or derivatives thereof. Exemplary hydroxycinnimac acids include caffeic acid, chlorogenic acid, coumaric acid, ferulic acid, or sinapic acid, and derivatives thereof. In another embodiment, fisetin or a derivative thereof is used. In yet another embodiment, baicalein, or a derivative thereof is used. It is understood that a polyphenol or flavonol can be administered in combination with other agents provided herein, or it may be administered independent of such agents.

In another embodiment, a formulation provided herein may include a polyphenol. The formulation is suitable for administration to a subject suffering from an embolic stroke.

The invention provides a method of treating stroke. The method includes contacting a subject suffering from a stroke with an antioxidant such as a polyphenol (e.g., chlorogenic acid) and/or a flavonol having antioxidant activity (e.g., fisetin and baicalein). The contacting is typically during a period of 5-60 minutes post stroke. In addition, the treatment therapy herein may be used in combination with other embolic stroke treatments. For example, following or concurrently with administration of the antioxidant a) an NMDA receptor antagonist; b) an agent that increases reperfusion of an affected area in an amount sufficient to allow for the penetration of a spin trap agent; and c) a spin trap agent in an amount sufficient to reduce cell and/or tissue damage can be administered.

In some embodiments, the reperfusion agent is a thrombolytic agent such as alteplase, tenecteplase, reteplase, streptase, abbokinase, pamiteplase, nateplase, desmoteplase, duteplase, monteplase, reteplase, lanoteplase, Prolyse™, microplasmin, Bat-tPA, BB-10153, or any combination thereof.

In other embodiments, the spin trap agent is a nitrone or nitroso spin trap compound. Such compounds include disodium 2,4-disulfophenyl-N-tert-butylnitrone (NXY-059), stilbazulenyl nitrone (STAZN), N-t-butyl-a-phenylnitrone, 3,5-dibromo-4-nitrosobenzenesulfonic acid, 5,5-dimethyl-1-pyrroline N-oxide, 2-methyl-2-nitrosopropane, nitrosodisulfonic acid, a-(4-pyridyl-1-oxide)-N-t-butylnitrone, 3,3,5,5-tetramethylpyrroline N-oxide, 2,4,6-tri-t-butylnitrosobenzene, PTIYO (4-phenyl-2,2,5,5-tetramethyl imidazolin-1-yloxy-5-oxide), tempol (4-hydroxy 2,2,6,6-tetramethylpiperidine-1-oxyl), or any combination thereof.

In some embodiments, the subject is contacted with the NMDA receptor antagonist prior to contact with the reperfusion agent and the spin trap agent and/or polyphenol antioxidant.

In other embodiments, the subject is contacted simultaneously with the reperfusion agent and the spin trap agent and/or polyphenol antioxidant.

In yet another embodiment, the subject is contacted with the reperfusion agent prior to contacting the subject with spin trap agent and/or polyphenol antioxidant.

In another embodiment, the NMDA receptor antagonist includes 3-alpha-ol-5-beta-pregnan-20-one hemisuccinate (ABHS), ketamine, memantine, dextromethorphan, dextrorphan, and dextromethorphan hydrobromide.

In some embodiments, the reperfusion agent is a thrombolytic agent and the NMDA receptor antagonist is either ABHS or memantine.

In other embodiments, the thrombolytic agent includes tPA or tNKA.

In one embodiment, a method of treating stroke that includes contacting a subject suffering from a stroke with an agent that increases reperfusion of an affected area, is provided. The method further includes contacting the subject with an NMDA receptor antagonist in an amount sufficient to reduce cell and/or tissue damage.

In another embodiment, a formulation including a thrombolytic agent and (i) an NMDA receptor antagonist, or (ii) a combination of an NMDA receptor antagonist and a spin trap agent, is provided. Exemplary thrombolytic agents include alteplase, tenecteplase, reteplase, streptase, abbokinase, pamiteplase, nateplase, desmoteplase, duteplase, monteplase, reteplase, lanoteplase, Prolyse™, microplasmin, Bat-tPA, BB-10153, and any combination thereof. Exemplary spin trap agents include disodium 2,4-disulfophenyl-N-tert-butylnitrone (NXY-059), N-t-butyl-a-phenylnitrone, stilbazulenyl nitrone (STAZN), 3,5-dibromo-4-nitrosobenzenesulfonic acid, 5,5-dimethyl-1-pyrroline N-oxide, 2-methyl-2-nitrosopropane, nitrosodisulfonic acid, a-(4-pyridyl-1-oxide)-N-t-butylnitrone, 3,3,5,5-tetramethylpyrroline N-oxide, 2,4,6-tri-t-butylnitrosobenzene, PTIYO (4-phenyl-2,2,5,5-tetramethyl imidazolin-1-yloxy-5-oxide), tempol (4-hydroxy 2,2,6,6-tetramethylpiperidine-1-oxyl), and any combination thereof. Exemplary NMDA receptor antagonists include 3-alpha-ol-5-beta-pregnan-20-one hemisuccinate (ABHS), ketamine, memantine, dextromethorphan, dextrorphan, and dextromethorphan hydrobromide.

In some embodiments, the formulation includes a thrombolytic agent that is tPA and an NMDA receptor antagonist that is ABHS.

In other embodiments, the formulation includes a thrombolytic agent that is tPA and an NMDA receptor antagonist that is memantine.

In yet another embodiment, the formulation includes a thrombolytic agent that is tNKA and an NMDA receptor antagonist that is ABHS.

In yet another embodiment, the formulation includes a thrombolytic agent that is tNKA and an NMDA receptor antagonist that is memantine.

The details of one or more embodiments of the disclosure are set forth in the accompanying drawings and the description below. Other features, objects, and advantages will be apparent from the description and drawings, and from the claims.

### BRIEF DESCRIPTION OF THE FIGURES

FIG. 1 is a graph depicting behavioral improvements following chlorogenic acid treatment. Chlorogenic acid administration resulted in significant behavioral improvement following embolic strokes when measured 24 hours following embolization or blood clot injection demonstrating that this antioxidant or synthetic derivatives of chlorogenic acid can be used to treat stroke.

FIG. 2 is a graph depicting behavioral improvements following fisetin treatment. Fisetin administration resulted in significant behavioral improvement following embolic strokes when measured 24 hours following embolization or blood clot injection demonstrating that this antioxidant or synthetic derivatives of fisetin can be used to treat stroke.

FIG. 3 depicts dose-response analysis for the effect of NXY-059 (0.10-100 mg/kg) administered 1 hour following embolization on behavioral outcome (P₅₀) measured 24 hours following embolism. NXY-059 at doses from 1-100 mg/kg significantly increased the P₅₀ (*p<0.05), but the lower dose did not (p>0.05). The data is presented as mean±SEM. The horizontal line represents the P₅₀ (in mg) for the vehicle group.

FIG. 4 depicts a therapeutic window for NXY-059 administration following embolic strokes. The curve shows that NXY-059 (100 mg/kg) administered 1 hour post-embolization results in a significant increase (*p<0.05) in P₅₀ measured in embolized rabbits. The P₅₀ measured in rabbits treated 3 or 6 hours following embolization is not significantly different from control values (p>0.05). The data is presented as mean±SEM. The horizontal line represents the P₅₀ (in mg) for the vehicle group.

FIG. 5 depicts percentage of rabbits behaviorally abnormal as a function of the amount of clots deposited into the brain (1-hour treatment time). The curve on the left (dotted) shows the response of vehicle-treated control rabbits to clot administration. It demonstrates that 50% of the animals treated with 1.20±0.15 mg of clots (P₅₀) are abnormal or dead 24 hours after injection of the clots. The curve in the middle (solid) indicates NXY-059 (100 mg/kg) increases the P₅₀ to 2.81±0.46 mg (*p<0.05). The dashed curve on right shows that Tenecteplase (0.9 mg/kg) significantly increases the P₅₀ to 2.76±0.37 mg (*p<0.05). The dashed-dotted curve shows that the combination of NXY-059 and Tenecteplase significantly increases the P₅₀ to 3.27±0.58 mg (p<0.05).

FIG. 6 depicts the percentage of rabbits behaviorally abnormal as a function of the amount of clots deposited into the brain (6 hour treatment time). The dotted and solid curves on the left show that neither Tenecteplase, nor NXY-059, significantly (p>0.05) affect P₅₀ values when given 6 hours following embolization compared to the vehicle control (i.e. 1.20±0.15 mg). The dashed curve on the right shows that co-administration of NXY-059 (100 mg/kg) and Tenecteplase (0.9 mg/kg) significantly increases the P₅₀ to 2.54±0.31 mg (p<0.05).

FIG. 7 depicts NMDA receptor antagonist ABHS increases the therapeutic window for tPA.

FIG. 8 depicts uncompetitive NMDA antagonist memantine increases the efficacy of low-dose tPA: synergy.

FIG. 9 depicts a Quantal analysis showing behavioral improvements following ABHS treatment. The control curve (dotted line) has a P₅₀ value of 1.15±0.19 mg (n=15). ABHS treatment (25 mg/kg) initiated 5 min following embolization increased the P₅₀ value to 2.60±0.69 mg (n=14, *P<0.05) (dark solid line). The closed circles represent the raw data from Table 1 for the control group, and the closed triangles represent the raw data for the ABHS-treated group. A normal animal for a specific clot weight is represented by a closed circle or a closed triangle symbol plotted at 0 on the y-axis, whereas an abnormal animal for a specific clot weight is represented by a closed circle or closed triangle symbol plotted at 100 on the y-axis.

FIG. 10 depicts the effect of combining ABHS with low-dose thrombolytic therapy on behavioral outcome following embolic strokes: synergism. Behavioral improvements following ABHS treatment in combination with low-dose tPA. The control curve (dotted line) has a P₅₀ value of 1.18±0.25 mg (n=24). Neither ABHS treatment (25 mg/kg; P₅₀=1.05±0.24 mg, n=17; light solid line) nor low-dose tPA treatment (0.9 mg/kg; P₅₀=1.25±0.25 mg, n=17; large dashed line) when initiated 60 min following embolization affected stroke-induced behavioral deficits, but the combination of ABHS (25 mg/kg) and tPA (0.9 mg/kg) increased the P₅₀ value to 2.44±044 mg (n=17, *P<0.05) (dark solid line).

FIG. 11 depicts behavioral improvements following treatment with a polyphenol (e.g., chlorogenic acid). The control curve (dotted line) has a P₅₀ value of 1.58±0.15 mg (n=26). Chlorogenic acid treatment (50 mg/kg IV) initiated 5 minutes following embolization increased the P₅₀ value to 3.61±0.52 mg (n=19, *P<0.05) (dark solid line).

FIG. 12 depicts exemplary chemical structures of polyphenols.

FIG. 13 is a graph showing behavioral improvements following chlorogenic acid (CGA) treatment. The cumulative control curve (dotted line) has a P₅₀ value of 1.58±0.15 mg (n=26). CGA treatment (50 mg/kg) initiated 5 minutes following embolization increased the P₅₀ value to 3.61±0.52 mg (n=19, *P<0.05) (dark solid line). The dark circles ● ▴ represent the raw data for the control group and the triangles A represent the raw data for the CGA-treated group. A normal animal for a specific clot weight is represented by a symbol plotted at 0 on the y-axis, whereas an abnormal animal for a specific clot weight is represented by a symbol plotted at 100 on the y-axis.

FIG. 14 shows the effect of CGA treatment on behavioral function when administered 5 minutes, 1 hour or 3 hours following embolization. CGA significantly increased behavior shown as the P₅₀ (mg of clot) when administered 5 minutes or 1 hour following embolization (p<0.05). CGA was ineffective at improving behavior when administered following a 3 hour delay (p>0.05).

FIG. 15 depicts behavioral improvements following CGA treatment in combination with tPA. The combination of CGA (50 mg/kg) and tPA(3.3 mg/kg) increased the P₅₀ value to 3.40±0.76 mg (*P<0.05, n=23) (dark solid line) when administered 3 hours following embolization. However, neither CGA treatment (50 mg/kg; light solid line, n=18) nor tPA treatment (3.3 mg/kg; large dashed line, n=18) when initiated 3 hours following embolization affected stroke-induced behavioral deficits. The dark circles represent the raw data for the CGA-tPA combination group. A normal animal for a specific clot weight is represented by a symbol plotted at 0 on the y-axis, whereas an abnormal animal for a specific clot weight is represented by a symbol plotted at 100 on the y-axis.

FIG. 16 shows behavioral improvements following baicalein treatment. The control curve (dotted line) has a P50 value of 1.37±0.20 mg (n=21). Baicalein treatment (100 mg/kg SC) initiated 5 minutes following embolization increased the P₅₀ value to 2.85±0.64 mg (n=21, *P<0.05) (dark solid line). The dark circles ● represent the raw data from the control group and the triangles ▴ represent the raw data for the Baicalein-treated group. A normal animal for a specific clot weight is represented by a symbol plotted at 0% on the y-axis, whereas an abnormal animal for a specific clot weight is represented by a symbol plotted at 100% on the y-axis.

### DETAILED DESCRIPTION

As used herein and in the appended claims, the singular forms “a,” “and,” and “the” include plural referents unless the context clearly dictates otherwise. Thus, for example, reference to “an antioxidant” includes a plurality of such antioxidants and reference to “the antioxidant” includes reference to one or more antioxidants known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

To date, all attempts at neuroprotection with single compounds, with the exception of Alteplase, which is not a neuroprotective, have been unsuccessful in acute ischemic stroke (AIS) patients. New therapies are needed. In addition to the need for new single therapeutic molecules, it is likely that combination therapies such as a thrombolytic with neuroprotective small molecules will be necessary to achieve optimal neuroprotection and clinical improvements following an AIS. Combining two or more drugs with differing mechanisms of action will afford maximal neuroprotection via additive or synergistic effects or provide additional benefit by increasing the therapeutic window for compounds or possibly by reducing side-effects. While there is a need to reduce the consequences of activation of the ischemic cascade, thrombolytics are valuable agents because they produce recanalization. This allows not only for reperfusion of ischemic tissue when the stroke is the result of a thrombus or embolus, but also provides improved access of small molecules, including drugs and nutrients to the penumbra of the infarcted tissue.

Although acute ischemic stroke is a treatable condition, using thrombolysis, which is the only approved method, current methods are far from perfect because of a short therapeutic window and significant concerns of intracerebral hemorrhage. Currently, treatment for acute ischemic stroke uses thrombolytic tissue plasminogen activator (tPA). Because of a narrow window of opportunity to treat a patient with tPA and significant side-effects associated with the use of tPA, the drug is underused. Even thrombolytic therapy with tPA produces complete resolution of symptoms less than 40% of the time, so there is need for additional forms of therapy. Numerous neuroprotective strategies have been tested in clinical trials, but none has been approved by the FDA for treating ischemic stroke.

In addition to the reduction of nutrients and oxygen to the site of ischemic injury, the process results in inflammatory damage and reactive oxygen species. For example, oxidative stress following ischemic stroke results in the creation of reactive oxygen species such as hydrogen peroxide (H₂O₂), hydroxyl radical (HO.) and superoxide anion radical (O2.) that cause lipid peroxidation and protein modification leading to neuronal and vascular damage and clinical deficits.

Ischemia also causes tissue damage resulting from the inflammatory cascade leading to leukotriene production via lipoxygenase activation. Lipoxygenases (LOXs) are dioxygenases that incorporate molecular oxygen into polyunsaturated fatty acids, such as arachidonic acid and, based on the site of insertion of the oxygen, are generally classified as 5-, 12-, or 15-LOXs. Recent evidence suggests that 12/15-LOX may play a role in ischemia-induced nerve cell loss. There is a correlation between an early reduction in GSH levels in ischemia and the activation of 12-LOX. Using in vitro cell culture assays, 12-LOX inhibitors have been shown to block glutamate-induced cell death and both 5- and 12-LOX inhibitors block ischemic injury in hippocampal slice cultures. Taken together, it appears that 12/15-LOX is an important enzyme that may mediate neurodegeneration following ischemia.

In one aspect, the invention allows for the treatment of patients following an embolic stroke and without, or with reduced, side-effects.

The methods and compositions of the invention include (1) an antioxidant; (2) an antioxidant and one or more of (i) a thrombolytic agent, (ii) an NMDA receptor antagonist and (iii) a spin trap agent; or (3) a thrombolytic agent in combination with one or more of (i) an NMDA receptor antagonist and (ii) a spin trap agent. Each of the foregoing are considered individually active agents in the methods and compositions of the invention.

The invention demonstrates that antioxidants including, but not limited to, chlorogenic acid, Baicalein and fisetin improve behavioral and survival rates of animal models of embolic stroke over a wide range of doses.

The invention provides a number of antioxidant therapies useful in treating oxidative stress associated with ischemic injury. For example, different antioxidants agents in various chemical classes: nitrones (STAZN), polyphenols, flavonols (e.g., baicalein) and phenylpropanoids (chlorogenic acid and fisetin), are useful in the methods of the invention as neuroprotection and to treat ischemic injury resulting from stroke.

The invention also contemplates the compositions, formulations and uses of chlorogenic acid compounds, Baicalein and/or fisetin alone or in combination with thrombolytic agents (e.g., Tenecteplase) and/or in combination with a spin trap agent (e.g., NXY-059) are useful as neuroprotectives. In performing these tests an embolic stroke model was used, which produces a behavioral endpoint to monitor and allows comparison of the efficacy of compounds. Not only was behavioral endpoints monitored by histochemical techniques were used to measure endpoints and pathology. The invention also provides data demonstrating the efficacy and safety of combining antioxidants with standard tPA therapy to determine if there are any positive or negative interactions when the drugs are combined. Thus, the invention provides data and evidence that STAZN, chlorogenic acid, baicalein and fisetin alter baseline hemorrhage measures and behavioral functions when administered alone or in combination with tPA. The data also demonstrate that these drugs are potentially effective as treatments for ICH.

Phenolic acids include, for example, caffeic acid, vanillin, and courmaric acid. Phenolic acids form a diverse group that includes the widely distributed hydroxybenzoic and hydroxycinnamic acids. Hydroxycinnamic acid compounds (p-coumaric, caffeic acid, ferulic acid) occur most frequently as simple esters with hydroxy carboxylic acids or glucose, while the hydroxybenzoic acid compounds (p-hydroxybenzoic, gallic acid, ellagic acid) are present mainly in the form of glucosides. Often phenolic acids occur as esters or glycosides conjugated with flavonoids, alcohols, hydroxyfatty acids, sterols, and glucosides.

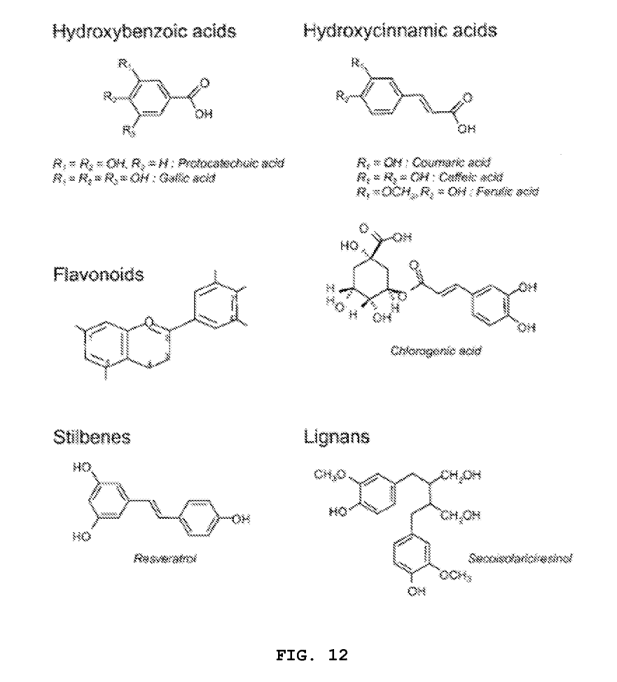

Several thousand molecules having a polyphenol structure (i.e., several hydroxyl groups on aromatic rings) have been identified in higher plants, and several hundred are found in edible plants. These molecules are secondary metabolites of plants and are generally involved in defense against ultraviolet radiation or aggression by pathogens. These compounds may be classified into different groups as a function of the number of phenol rings that they contain and of the structural elements that bind these rings to one another. Distinctions are thus made between the phenolic acids, flavonoids, stilbenes, and lignans (FIG. 12). In addition to this diversity, polyphenols may be associated with various carbohydrates and organic acids and with one another (see Manach et al., Am. J. Clin. Nutr., 79:727-747 (2004), incorporated herein by reference).

The hydroxycinnamic acids are more common than are the hydroxybenzoic acids and consist chiefly of p-coumaric, caffeic, ferulic, and sinapic acids. These acids are rarely found in the free form, except in processed food that has undergone freezing, sterilization, or fermentation. The bound forms are glycosylated derivatives or esters of quinic acid, shikimic acid, and tartaric acid. Caffeic and quinic acid combine to form chlorogenic acid, which is found in many types of fruit and in high concentrations in coffee: a single cup may contain 70-350 mg chlorogenic acid. The types of fruit having the highest content (blueberries, kiwis, plums, cherries, apples) contain 0.5-2 g hydroxycinnamic acids/kg fresh wt.

Caffeic acid, both free and esterified, is generally the most abundant phenolic acid and represents between 75% and 100% of the total hydroxycinnamic acid content of most fruit. Hydroxycinnamic acids are found in all parts of fruit, although the highest concentrations are seen in the outer parts of ripe fruit. Concentrations generally decrease during the course of ripening, but total quantities increase as the fruit increases in size.

Ferulic acid is the most abundant phenolic acid found in cereal grains, which constitute its main dietary source. The ferulic acid content of wheat grain is ˜0.8-2 g/kg dry wt, which may represent up to 90% of total polyphenols. Ferulic acid is found chiefly in the outer parts of the grain. The aleurone layer and the pericarp of wheat grain contain 98% of the total ferulic acid. The ferulic acid content of different wheat flours is thus directly related to levels of sieving, and bran is the main source of polyphenols. Rice and oat flours contain approximately the same quantity of phenolic acids as wheat flour (63 mg/kg), although the content in maize flour is about 3 times as high. Ferulic acid is found chiefly in the trans form, which is esterified to arabinoxylans and hemicelluloses in the aleurone and pericarp. Only 10% of ferulic acid is found in soluble free form in wheat bran. Several dimers of ferulic acid are also found in cereals and form bridge structures between chains of hemicellulose.

An exemplary polyphenol includes the phenolic acid chlorogenic acid. Chlorogenic acid may be chemically synthesized. Alternatively, is may be isolated from the leaves and fruits of dicotyledonous plants, including the coffee bean. Structurally, chlorogenic acid is the ester of caffeic acid with the 3-hydroxyl group of quinic acid (CAS RN: 327-97-9; formula: C16H18O9; Melting point ° C.: 207-209; MW: 354.3128). Synonyms include for chlorogenic acid include 3-[[3-(3,4-Dihydroxyphenyl)-1-oxo-2-propenyl]oxy]1,4,5-trihydroxycyclohexanecarboxylic acid. Chlorogenic acid exists predominantly trans in nature (e.g., coffee seeds).

Flavonoids are a subclass of polyphenols. The polyphenolic structure of flavonoids and tannins renders them quite sensitive to oxidative enzymes and cooking conditions.

Catechins or Flavanols are found in teas and grapes and include, for example, monomeric flavan-3-ols catechin, epicatechin, gallocatechin, epigallocatechin, and epicatechin 3-O-gallate. Grape seed extract comprise polyphenols including, for example, proanthocyanidins. Proanthocyanidins share common properties with other polyphenols, in particular their reducing capacity and ability to chelate metal ions. The most common flavonols in the diet are Quercetin, Kaempferol, and Myricetin. Flavonols also include fisetin, isoquercitrin and hyperoside.

Chlorogenic acid is a phenolic natural product isolated from the leaves and fruits of dicotyledonous plants, including the coffee bean. Structurally, chlorogenic acid is the ester of caffeic acid with the 3-hydroxyl group of quinic acid. Fisetin is a natural flavonol present in edible vegetables, fruits and wine. These compounds are generally described as antioxidants and kinase inhibitors. The invention demonstrates that such naturally occurring compounds are useful to treat embolic stroke and to enhance the therapeutic window for treatment with other ischemic stroke agents such as spin trap agents and NMDA inhibitors. Other flavonols that can be used in the methods and compositions of the invention include, but are not limited to, troxerutin, venoruton, hydroxyethylrutosides, hesperitin, naringenin, nobiletin, tangeritin, baicalein, galangin, genistein, quercetin, apigenin, kaempferol, fisetin, rutin, luteolin, chrysin, taxifolin, eriodyctol, catecithin, epicatechin, epigallocatechin, epicatechin gallate, epigallocatechin gallate, flavone, sideritoflavone, hypolaetin-8-O-Gl, oroxindin, 3-hydroxyflavone, morin, quercetagetin-7-O-Gl, tambuletin, gossypin, hipifolin, naringin, leucocyanidol, amentoflavone and derivatives thereof and mixtures thereof. (−)-epigallocatechin; (−)-epigallocatechin-gallate; 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; catechin; chrysin; chrysin-5,7-dihydroxyflavone; chrysoeriol; chrysosplenol; chrysosplenoside-a; chrysosplenoside-d; cosmosiin; 6-cadinene; curcumin; dihydroquercetin; dimethylmussaenoside; diacerylcirsimaritin; diosmetin; dosmetin; ellagic acid; ebinin; epicatechin; ethyl brevifolin carboxylate; flavocannibiside; flavosativaside; genistein; ginkgo flavone glycosides; ginkgo heterosides; gossypetin; gossypetin-8-glucoside; haematoxylin; hesperidine; hispiduloside; hyperin; indole; iridine; isoliquiritigenin; isoliquiritin; isoquercitrin; jionoside; juglanin; kaempferol; kaempferol-3-rhamnoside; kaempferol-3-neohesperidoside; kolaviron; licuraside; linariin; linarin; lonicerin; luteolin; luteolin-7-glucoside; luteolin-7-glucoronide; macrocarpal-a; macrocarpal-b; macrocarpal-d; macrocarpal-g; maniflavone; morin; methyl scutellarein; myricetin; naringenin; naringin; nelumboside; nepetin; nepetrin; nerolidol; oligomeric proanthocyanidins; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; silibin; silydianin; silychristine; silymarin; sophoricoside; sorbarin; spiraeoside; trifolin; vitexin; wogonin; and pharmaceutically acceptable salts of any of the foregoing.

Baicalein (5,6,7 trihydroxyflavone) is also a 12/15-LOX inhibitor that reduces neutrophil-mediated inflammatory reactions in rat brain ischemia. Baicalein is a potent free radical scavenger and xanthine oxidase inhibitor. In addition to 12-LOX, baicalein has a weak inhibitory effect on 5-LOX and leukotriene synthesis. It has, in fact, been observed that many flavonoids have the ability to inhibit both LOX and COX subtypes.

The disclosure also provides ways that antioxidants can be analyzed to determine their ability to reduce neurological damage caused by embolic strokes by measuring oxidative DNA damage, peroxynitrite-derived lipid peroxidation and oxidative protein damage (protein carbonylation). The mechanisms identified herein provide a basis for the development of therapeutics using, for example, a proteomics technique to identify signaling pathways that mediate cell damage associated with ischemic stroke and determine the effects of the polyphenol antioxidants in treating ischemia-associated damage. Thus, the invention also provides methods and techniques useful to identify pathways involved in neuroprotection and new molecular targets for future therapies.

Nitrone-based spin trap agents such as NXY-059 and thrombolytics, such as Tenecteplase, are currently being developed for the treatment of acute ischemic stroke (AIS) since they are two of the most promising drug candidates.

Spin traps such as nitroxides and nitrones are stabilized forms of the biological messenger nitric oxide. Unlike other antioxidants, spin traps neither act as proxidants, nor do they propagate free radical chain reactions. Likewise, these agents inhibit the reaction of superoxide and nitric oxide to produce peroxinitrite. Thus, combination therapies with spin traps and therapeutic agents currently under development or in use for such diseases and disorders as Parkinsonism, stroke, ischaemic injury, heart attack, and age-related dementias are encompassed by the invention.

Nitrone and nitroso spin trap compounds are commercially available. Exemplary nitrone and nitroso spin trap compounds include disodium 2,4-disulfophenyl-N-tert-butylnitrone (NXY-059), N-t-butyl-α-phenylnitrone, 3,5-dibromo-4-nitrosobenzenesulfonic acid, 5,5-dimethyl-1-pyrroline N-oxide, 2-methyl-2-nitrosopropane, nitrosodisulfonic acid, α-(4-pyridyl-1-oxide)-N-t-butylnitrone, 3,3,5,5-tetramethylpyrroline N-oxide, 2,4,6-tri-t-butylnitrosobenzene, PTIYO (4-phenyl-2,2,5,5-tetramethyl imidazolin-1-yloxy-5-oxide) and tempol (4-hydroxy 2,2,6,6-tetramethylpiperidine-1-oxyl) and the like.

Preclinical studies with NXY-059 have suggested that the spin trap may be beneficial for the treatment of stroke because it reduces infarct volume following middle cerebral artery (MCA) occlusion in rodents and primates and produces some behavioral improvement in both species. Moreover, spin trap agents (e.g., NXY-059) can improve clinical rating scores if administered to rabbits following small clot embolic strokes and can increase the therapeutic window for thrombolytic agents. Recent reports indicate that NXY-059 traps carbon- and oxygen-centered radicals in solution (Maples et al.). It is hypothesized that the beneficial effect of spin trap agents such as NXY-059 in the rabbit embolic stroke model, as well as the rodent and primate stroke models, is due to suppression of the cascade activated by free radicals. This may include the reduction of lipid oxidation and both microvascular and mitochondrial dysfunction. Moreover, since NXY-059 probably does not cross the blood brain barrier to any appreciable extent for the first 6 hours after a stroke, it is probably producing its effects in the microvasculature.

Any number of thrombolytic agents can be used in the methods and compositions of the invention. Examples of thrombolytic agents that can be use in the methods and composition of the invention include alteplase, tenecteplase, reteplase, streptase, abbokinase, pamiteplase, nateplase, desmoteplase, duteplase, monteplase, reteplase, lanoteplase, and Prolyse™). Other thrombolytics include, for example, microplasmin, Bat-tPA, BB-10153 (an engineered form of human plasminogen activated to plasmin by thrombin) and Desmodus rotundus salivary plasminogen activators (DSPAs) (e.g., DSPAα1).

In contrast to the information available for NXY-059, there are only two preclinical studies with Tenecteplase, both which show that Tenecteplase can improve behavioral deficits following embolic strokes. Thus, NXY-059 and Tenecteplase are prime candidates for continued development at both the preclinical and clinical levels.

The invention demonstrates that a spin trap agent (e.g., NXY-059) improved behavioral performance over a wide range of doses following embolization. However, if administered at a long delay (>1 hour) following embolization the neuroprotective activity or increase in behavioral improvement was lost. Furthermore, the invention demonstrates that administration of a spin trap agent (e.g., NXY-059) in combination with a thrombolytic agent (e.g., Tenecteplase) 1 or 6 hours following embolization was safe, and there was a statistically significant synergistic effect of the drug combination on clinical rating scores when administered 6 hours after a stroke.

For example, the spin trap agent NXY-059 significantly reduced embolism-induced behavioral deficits when administered within 1 hour of embolization. However, if drug administration was delayed to 3 or 6 hours, behavioral improvements were no longer observed and the P₅₀ values were not statistically different from the vehicle group. This neuroprotective effect of NXY-059 is in agreement with a variety of previous studies. However, the observation that NXY-059 has a short therapeutic window following embolic strokes in rabbits is different from that published by various other groups using MCAO occlusions. All three studies, show that NXY-059 is neuroprotective if given between four and six hours after the stroke, if administered using a bolus loading dose followed by long-term infusion. The differences between these results and those by other investigators may be related to the treatment regimen used in the studies (short infusions vs. bolus injections/long-term infusions) and the method of stroke induction. Accordingly, various methods of delivery/infusion are encompassed by the invention. Nevertheless, all studies are in agreement with the basic finding, that is, NXY-059 produces significant behavioral improvement following an acute ischemic stroke.

Moreover, administration of the combination of drugs was safe, no negative behavioral consequences of administration of the combination of drugs was observed. For example, previous studies have shown that Tenecteplase had a 3 hour therapeutic window. When a spin trap agent was administered in combination with a thrombolytic (e.g., Tenecteplase) starting 1 hour following embolization, there was a significant, but not additive or synergistic effect of the drug combination on behavioral rating scores. The lack of an additive or synergistic effect was not completely unexpected since both drugs were administered at maximally effective doses at a time point where they each produce significant behavioral improvement. However, if the spin trap agent (e.g., NXY-059) and the thrombolytic (e.g., Tenecteplase) were administered concomitantly 6 hours following embolization, the drug combination significantly increased the P₅₀ value. Furthermore, the P₅₀ value measured for the drug combination group was significantly different from that measured for NXY-059 when administered 6 hours following embolization. This result indicated that the behavioral effect of the drug combination administered following the therapeutic window of Tenecteplase (e.g., at about 6 hours) was similar in magnitude to either NXY-059 or Tenecteplase alone when administered 1 hour following embolization. Results with the combination of NXY-059 and Tenecteplase are similar to those for NXY-059 and Alteplase. For example, pretreatment with NXY-059 prior to Alteplase administration increased the therapeutic window for Alteplase. Since pretreatment with a spin trap agent is unlikely in patients, the current study used concomitant administration of drugs at lengthy delays (1-6 hours) following the stroke to attempt to better represent the clinical situation. The observation that the combination of a spin trap agent and a thrombolytic (e.g., NXY-059 and Tenecteplase, or NXY-059 and Alteplase) was effective from about 1 to about 6 hours following embolization demonstrating that this combination treatment is a promising approach to treat AIS. Since NXY-059 significantly reduces embolization-induced behavioral deficits at the doses used in the study, NXY-059 appears to effectively scavenge free radicals produced during and following an ischemic stroke induced by injection of blood clots.

The data show that the spin trap agent, NXY-059, is effective as a monotherapy for AIS. However, the short therapeutic window for NXY-059 following an embolic stroke presents significant difficulties for treating patients, who normally do not present until several hours following a stroke. The results with combination therapies are promising for a number of reasons. Since two drugs with differing pharmacological properties will be administered, significant benefits from the mechanism of action of each independent drug may be produced. The application of a thrombolytic will allow for reperfusion of poorly perfused tissues and allow for the penetration of a spin trap agent, oxygen and glucose into the penumbra of ischemic tissue to scavenge free-radicals. By virtue of the mechanism of action of spin trap agents such as NXY-059, the deleterious effect of free radicals may be attenuated.

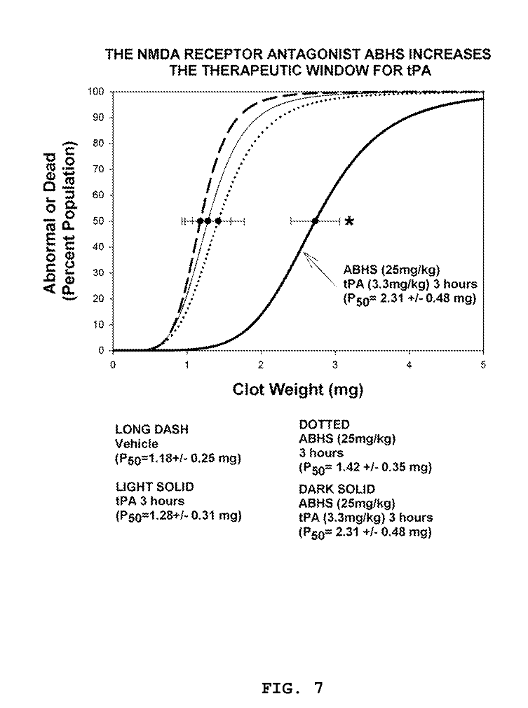

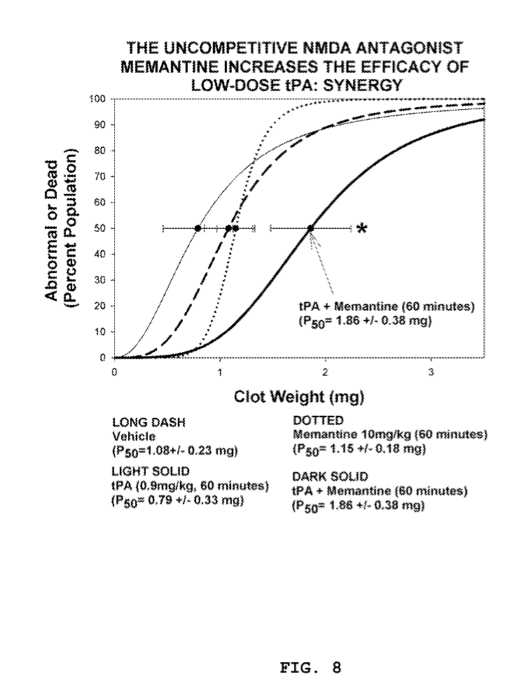

In addition to the generation of free radicals, early response in an ischemic event is the rapid release of excitatory amino acids followed by the activation of the “ischemic cascade”. It has been suggested that neurosteroids, which act as negative modulators of excitatory amino acid receptors, may improve behavioral functions and promote neuronal survival following ischemia. The data (see, FIGS. 7 and 8) demonstrate the pharmacological effects of 3-alpha-ol-5-beta-pregnan-20-one hemisuccinate (ABHS), a neurosteroid that inhibits excitatory amino acid receptor function increases the therapeutic window for thrombolytic agents.

Accordingly, in another aspect of the invention, methods and formulations comprising an NMDA (N-Methyl-D-Aspartate) receptor antagonists are provided that, in combination with a thrombolytic agent improved behavioral performance. The NMDA antagonist can also be combined with a thrombolytic agent and a spin trap agent or an antioxidant (e.g., a chlorogenic agent, fisetin, baicalein). Examples of NMDA receptor antagonists include 3-alpha-ol-5-beta-pregnan-20-one hemisuccinate, ketamine, memantine, dextromethorphan, dextrorphan, and dextromethorphan hydrobromide. Piperidine derivatives and analogues substituted with phenols or phenol equivalents having NR2B selective NMDA antagonist activity are described in international patent application nos. WO 90/14087, WO 90/14088, WO 97/23202, WO 97/23214, WO 97/23215, WO 97/23216, WO 97/23458, WO 99/21539, WO 00/25109, European patent application No. EP 648744 A1 and in U.S. Pat. No. 5,436,255. Compounds containing 2-benzoxazolinone substructure with the same biological activity are described in international patent applications WO 98/18793 and WO 00/00197. Other NR2B selective NMDA antagonists having condensed heterocyclic structures are described in international patent application nos. WO 01/30330, WO 01/32171, WO 01/32174, WO 01/32177, WO 01/32179, 01/32615, WO 01/32634.

In a rabbit reversible spinal cord ischemia model (RSCIM). ABHS was administered (25 mg/kg) intravenously (i.v.) 5 or 30 min following the start of occlusion to groups of rabbits exposed to ischemia induced by temporary occlusion of the infrarenal aorta. The group P₅₀ represents the duration of ischemia (min) associated with a 50% probability of resultant permanent paraplegia. Quantal analysis indicated that the P50 of the control group was 23.44+/−4.32 min. Using the RSCIM, neuroprotection is observed if a drug significantly prolongs the P₅₀ compared to the control group. Treatment with ABHS (25 mg/kg) 5 min post-occlusion significantly (p<0.05) prolonged the P₅₀ of the group to 49.18+/−10.44 min, an increase of 110%. If ABHS was injected 5 min following the start of ischemia and again 24 h after ischemia, there was a persistent effect of the drug at 48 h. Moreover, ABHS also increased the tolerance to ischemia if administered 30 min following the start of occlusion. The results suggest that neuroactive steroids such as ABHS, which are selective NMDA receptor antagonists, may have substantial therapeutic benefit for the treatment of ischemic injuries including spinal cord neurodegeneration and stroke.

The invention provides methods of treating stroke and/or ischemic injury comprising administering to a subject a polyphenol, flavonoid, or flavonol antioxidant (e.g., a chlorogenic agent, fisetin or baicalein). In a further aspect, the invention comprises administering an antioxidant and (i) a thrombolytic agent, (ii) a spin trap agent, (iii) an NMDA receptor antagonist, or (iv) any combination of (i), (ii) and (iii).

Active agents of the invention (e.g., a polyphenol antioxidant, a spin trap agent, an NMDA receptor antagonist, and a thrombolytic agent) are typically employed in the form of a pharmaceutical formulation. Conveniently, spin trap agents and a thrombolytic agents may be presented together in a single formulation rather than using separate formulations for each. Accordingly, the invention provides a pharmaceutical formulation, which comprises an antioxidant, and may further comprise a spin trap agent and/or an NMDA receptor antagonist, and a thrombolytic agent and a pharmaceutically acceptable carrier.

As used herein, the term “an ischemic injury alleviating amount” or “effective amount” means the amount of a composition comprising an antioxidant, and in some embodiments, a spin trap agent and/or an NMDA receptor antagonist, and a thrombolytic agent useful for causing a diminution in ischemic injury, whether by alleviating free-radical damage, alleviating behavioral changes, or by promoting reperfusion of the damaged tissue. An effective amount to be administered systemically depends on the body weight of the subject. Typically, an effective amount to be administered systemically is about 0.1 mg/kg to about 100 mg/kg. An effective amount of an antioxidant (e.g., a polyphenol such as chlorogenic acid, baicalein or fisetin) alone or in combination with a spin trap/NMDA receptor antagonist and a thrombolytic formulation to inhibit damage to jeopardized tissue will of course depend upon a number of factors including, for example, the age and weight of the subject (e.g., a mammal such as a human), the precise condition requiring treatment and its severity, the route of administration, and will ultimately be at the discretion of the attendant physician or veterinarian.

Typically an effective amount of a formulation of the invention is injected directly into the bloodstream of the subject. For example, intravenous injection can be used to administer the formulation to the peripheral or central nervous system because the formulation is capable of crossing the blood-brain barrier and entering the central nervous system.

Oral administration often can be desirable, provided the formulation is modified so as to be stable to gastrointestinal degradation and readily absorbable.

Direct intracranial injection or injection into the cerebrospinal fluid also can be used to introduce an effective amount of a thrombolytic agent and a spin trap agent, and alternatively an NMDA antagonist. In some embodiments, an antioxidant alone or in combination with a spin trap and/or thrombolytic agent can be administered into the central nervous system of a subject. In addition, an antioxidant alone or in combination with a spin trap/NMDA receptor antagonist and thrombolytic agent can be administered to peripheral neural tissue by direct injection or local topical application or by systemic administration. Various conventional modes of administration also are contemplated, including intravenous, intramuscular, intradermal, subcutaneous, intracranial, epidural, topical, oral, transdermal, transmucosal, and intranasal administration.

Any of the formulations of the invention can be administered in a sustained release form. The sustained release formulation has the advantage of delivery over an extended period of time without the need for repeated administrations of the formulation.

Sustained release can be achieved, for example, with a sustained release material such as a wafer, an immunobead, a micropump or other material that provides for controlled slow release of the antioxidant or combination formulation. Such controlled release materials are well known in the art and available from commercial sources. In addition, a bioerodible or biodegradable material can be formulated with active agents of the invention, such as polylactic acid, polygalactic acid, regenerated collagen, multilamellar liposomes or other conventional depot formulations, can be implanted to slowly release the antioxidant, thrombolytic, spin trap, and NMDA antagonist agents. The use of infusion pumps, matrix entrapment systems, and transdermal delivery devices also are contemplated in the invention.

Active agents/formulations also can be advantageously enclosed in micelles or liposomes. Liposome encapsulation technology is well known. Liposomes can be targeted to a specific tissue, such as neural tissue, through the use of receptors, ligands or antibodies capable of binding to an antigen or target in the desired tissue. The preparation of these formulations is well known in the art (see, for example, Pardridge, supra (1991), and Radin and Metz, Meth Enzymol. 98:613-618 (1983)).

A composition/formulation of the invention can be packaged and administered in unit dosage form, such as an injectable composition/formulation or local preparation in a dosage amount equivalent to the daily dosage administered to a subject, and if desired can be prepared in a controlled release formulation. Unit dosage form can be, for example, a septum sealed vial containing a daily dose of an antioxidant, and in another embodiment, a combination of a spin trap/thrombolytic formulation of the invention in PBS or in lyophilized form. For treatment of neural damage associated with, for example, acute ischemic stroke, an appropriate daily systemic dosage of an antioxidant formulation (e.g., comprising chlorogenic agent, fisetin, baicalein or a combination of a spin trap/thrombolytic formulation) is based on the body weight of the subject and is in the range of from about 0.1 ug/kg to about 100 mg/kg, although dosages from about 0.1 mg/kg to about 100 mg/kg are also contemplated. Thus, for the typical 70 kg human, a systemic dosage can be between about 7 ug and about 7,000 mg daily. A daily dosage of locally administered material will be about an order of magnitude less than the systemic dosage. Oral administration is also contemplated.

Generally, an antioxidant (e.g., a chlorogenic agent, fisentin or baicelain), which may be in combination with a spin trap agent and/or an NMDA receptor antagonist, and a thrombolytic agent will be administered by the intravascular route and thus a parenteral formulation is used. Typically a lyophilized formulation is used by the physician or veterinarian because of the significant transportation and storage advantages that it affords. The physician or veterinarian may then reconstitute the lyophilized formulation in an appropriate amount of solvent as and when required.

Parenteral and lyophilized pharmaceutical formulations containing thrombolytics are known in the art (see, e.g., EP-A-41 766, EP-A-93 619, EP-A-112 122, EP-A-113 319, EP-A-123, EP-A-113 319, EP-A-123 304, EP-A-143 081, EP-A-156 169, Japanese patent publication 57-120523 (application No. 56-b 6936) and Japanese patent publication 58-65218 (Application no. 56-163145). Additional examples include UK patent applications Nos. 8513358, 8521704 and 8521705. All such formulations are also suitable for and antioxidant, a spin trap and for the combination of any of the above with an NMDA receptor antagonist, and a thrombolytic agent.

Intravascular infusions are normally carried out with the parenteral solution contained within an infusion bag or bottle or within an electrically operated infusion syringe. The solution may be delivered from the infusion bag or bottle to the subject by gravity feed or by the use of an infusion pump. The use of gravity feed infusion systems in some instances does not afford sufficient control over the rate of administration of the parenteral solution and, therefore, the use of an infusion pump may be desirable especially with solutions containing relatively high concentrations of spin trap/thrombolytic formulation. An electrically operated infusion syringe may offer even greater control over the rate of administration.

The invention also provides a method for inhibiting damage to jeopardized tissue during reperfusion in a mammal, which comprises administering to the mammal an effective amount of an antioxidant (e.g., a polyphenol such as chlorogenic acid, baicalein or fisetin) alone or in combination with a spin trap/NMDA receptor antagonist and a thrombolytic formulation. In the alternative, the invention provides a combination of an antioxidant, spin trap agent and/or an NMDA receptor antagonist, and a thrombolytic agent for use in human and veterinary medicine especially for use in inhibiting damage to jeopardized tissue during reperfusion in a mammal. It will be understood that the administration of an antioxidant can be made simultaneously with a spin trap agent and/or an NMDA receptor antagonist, and a thrombolytic agent; however, in some instances, one may be administered either before during or after the other.

The agents may be administered simultaneously or sequentially in separate formulations or may be administered simultaneously in a single formulation. In any event the delay in administering the second or third etc. of a plurality of agents should not be such as to lose the benefit of a potentiated effect of the combination of the agents in vivo in inhibiting tissue damage. For example, where a single formulations comprising an antioxidant and one or more additional therapeutic agents (e.g., a spin trap/thrombolytic agent) are not available, administration of a composition comprising an antioxidant may be performed followed by administration of a thrombolytic agent, spin trap agent and/or NMDA antagonist, or vice versa.

The invention is particularly advantageous in inhibiting damage to jeopardized tissue arising from the occurrence of a blood clot in that, as mentioned previously, both the removal of the blood clot and the protection of the jeopardized tissue can be achieved.

Formulations will typically comprise (1) an antioxidant; (2) an antioxidant and one or more of (i) a thrombolytic agent, (ii) an NMDA receptor antagonist and (iii) a spin trap agent; or (3) a thrombolytic agent in combination with one or more of (i) an NMDA receptor antagonist and (ii) a spin trap agent, in a pharmaceutically acceptable carrier. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by a subject.

Pharmaceutical preparations for oral use can be obtained through combination of active agents (e.g., (1) an antioxidant; (2) an antioxidant and one or more of (i) a thrombolytic agent, (ii) an NMDA receptor antagonist and (iii) a spin trap agent; or (3) a thrombolytic agent in combination with one or more of (i) an NMDA receptor antagonist and (ii) a spin trap agent) with a solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical formulations that can be used orally include push-fit capsules made of gelatin, as well as soft, sealed capsules made of gelatin and a coating such as glycerol or sorbitol. Push-fit capsules can contain active agent mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active agent ((1) an antioxidant; (2) an antioxidant and one or more of (i) a thrombolytic agent, (ii) an NMDA receptor antagonist and (iii) a spin trap agent; or (3) a thrombolytic agent in combination with one or more of (i) an NMDA receptor antagonist and (ii) a spin trap agent). For injection, the pharmaceutical compositions of the invention may be formulated in aqueous solutions, typically in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the formulation may also contain suitable stabilizers or agents which increase the solubility of the compounds to allow for the preparation of highly concentrated solutions.

The following example is provided in illustration of the present invention and should not be construed in any way as constituting a limitation thereof.

### EXAMPLES

The invention used a rabbit small clot embolic stroke model (RSCEM) that reproduces many facets of human AIS including a well-defined clinical endpoint. The RSCEM utilizes administration of a suspension of small blood clots to induce strokes, and behavioral deficits that can be measured quantitatively. Moreover, the RSCEM is useful to conduct studies testing the effects of single drugs or drug combinations such as a neuroprotective agent (e.g., NXY-059) plus a thrombolytic (Tenecteplase, Alteplase, Desmoteplase and the like). Since it is likely that a drug combination will be used for patient management it is important to determine whether there are any interactions (positive or negative) between the compounds when administered following embolic strokes.

Methods: Male New Zealand white rabbits were anesthetized using halothane (5% induction, 2% maintenance by facemask), the bifurcation of the right carotid artery was exposed and the external carotid was ligated just distal to the bifurcation, where a catheter was inserted into the common carotid and secured with ligatures. The incision was closed around the catheter with the distal ends left accessible outside the neck; the catheter was filled with heparinized saline and plugged with an injection cap. Rabbits were allowed to recover from anesthesia for a minimum of 3 h until they awoke and behaved normally.

For the RSCEM, microclots were prepared from blood drawn from a donor rabbit and allowed to clot at 37° C. All rabbits in the study received injections of the same sized microclots. For embolization, clot particles were rapidly injected through the carotid catheter and both the syringe and catheter were flushed with 5 ml of normal saline.

Drug Administration: The spin trap agent NXY-059 was custom-synthesized by Dr. Robert Purdy (VASDHS, San Diego, Calif.) according to the synthetic scheme used previously. The proton magnetic resonance spectrum at 550 Hz of the pure white crystalline product in deuterochloroform was consistent with disodium-[(tert-butylimino)methyl]benzene-1,3-disulfonate N-oxide (i.e.: NXY-059). Genentech, Inc. (South San Francisco, Calif.) supplied Tenecteplase as a lyophilized cake, which is in the same formulation used clinically. It was reconstituted with sterile water prior to injection.

Treatment Regimens: (A) Dose-Response Curve: NXY-059 was administered IV at a dose of 0.1-100 mg/kg infused over 30 min beginning 1 hour following embolization. (B) Therapeutic Window: NXY-059 (100 mg/kg) was given beginning 1, 3 or 6 hours following embolization. (C) Tenecteplase Dosing: Tenecteplase was administered IV at a dose of 0.9 mg/kg as a bolus injection given over one minute was administered 1 or 6 hours following embolization. (D) Combination studies: NXY-059 was administered IV at a dose of 100 mg/kg infused over 30 min and Tenecteplase was administered IV at a dose of 0.9 mg/kg beginning 1 or 6 hours following embolization.

Quantal Dose-Response Analysis: For the RSCEM, a quantal dose-response data analysis technique was used. A wide range of lesion volumes is induced to generate both normal and abnormal animals. Using 3 or more different doses of microclots generated each quantal analysis curve. In the absence of neuroprotective compounds or thrombolytics, the data show the low end of the curve (small numbers of microclots cause no grossly apparent neurologic dysfunction) and the high end (large numbers of microclots invariably cause encephalopathy or death). Each animal is rated as either normal or abnormal (including dead animals), and inter-rater variability is very low (<5%). Behaviorally normal rabbits did not have any signs of impairment, whereas behaviorally abnormal rabbits had loss of balance, head leans, circling, seizure-like activity, or limb paralysis. With this simple rating system, the composite result for a group of animals is quite reproducible. The P₅₀ values are then calculated. These parameters are measures of the amount of microclots (in mg) that produce neurologic dysfunction in 50% of a group of animals. A separate curve is generated for each treatment condition that was tested. The data were analyzed using the t-test, which included the Bonferroni correction where appropriate.

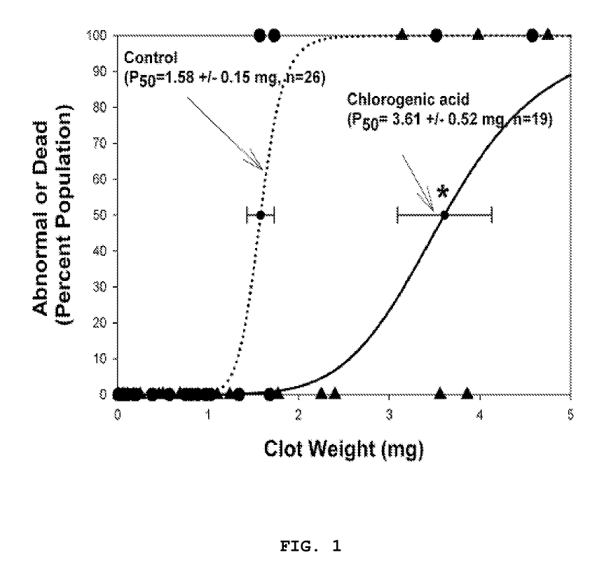

1) NXY-059 Dose-Response Profile: In the RSCEM, the P₅₀ for vehicle-treated embolized rabbits measured 24 hours after embolization was 1.20±0.15 mg (n=18) (FIG. 1). When varying doses of NXY-059 were administered 1 hour following embolization, the P₅₀ was between 1.60±0.27 mg and 2.81+0.46 mg, with doses between 1.0 mg/kg and 100 mg/kg producing significant behavioral improvements (p<0.05 compared to vehicle) when measured 24 hours after embolization.

(2) NXY-059 Therapeutic Window: In the RSCEM, NXY-059 administration 1 hour post embolization significantly increased the P₅₀ value as described above (FIG. 1). However, NXY-059 was no longer effective when administered either 3 hours or 6 hours following embolization since the P₅₀ values were 1.78±0.42 mg (n=16) and 1.35±0.37 mg (n=20), respectively (p>0.05 compared to vehicle).

(3) Tenecteplase Administration: Tenecteplase improves behavioral in embolized rabbits when administered at doses between 0.9 mg/kg and 3.3 mg/kg starting 1 hour following embolization. In this study, Tenecteplase administered at 0.9 mg/kg also significantly improved behavioral outcome, since the P₅₀ values was 2.76±0.37 mg (n=15), an increase of 130% over control. However, when there was a 6 hour delay before Tenecteplase treatment, it did not increase the P₅₀ compared to control.

(4) Combination therapies: Since both NXY-059 (100 mg/kg) and Tenecteplase (0.9 mg/kg) were effective when administered 1 hour following embolization, respectively, tests were performed to determine whether administering both drugs in combination starting 1 hour following embolization would be safe and provide additional behavioral improvement (i.e. a further increase in the P₅₀ value compared to either drug alone). In the combination-treated group, the P₅₀ value was 3.27±0.58 mg (n=18, p<0.05 compared to vehicle, p>0.05 compared to either NXY-059 or Tenecteplase), an increase of 16% and 18% for NXY-059 and Tenecteplase, respectively). However, when NXY-059 (100 mg/kg) and Tenecteplase were administered together 6 hours following embolization, the P₅₀ value was 2.54±0.31 mg (p<0.05 compared to vehicle or NXY-059 alone at 6 hours, p>0.05 compared to Tenecteplase at 6 hours). The combination of NXY-059 and Tenecteplase was statistically better than NXY-059 alone at 6 hours.

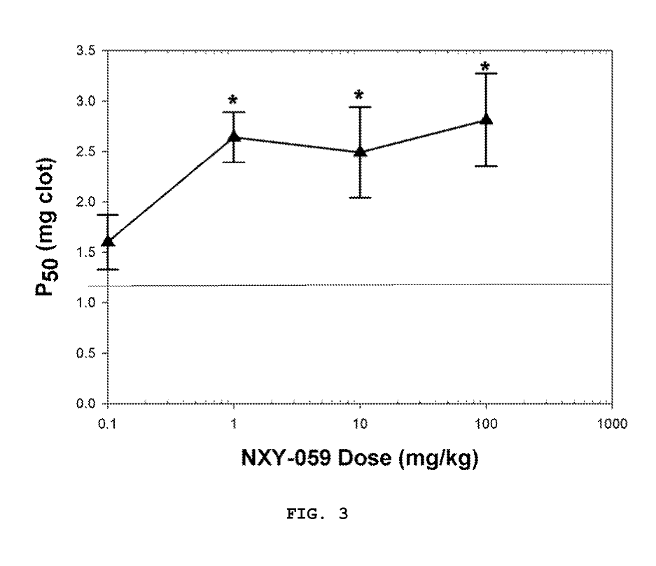

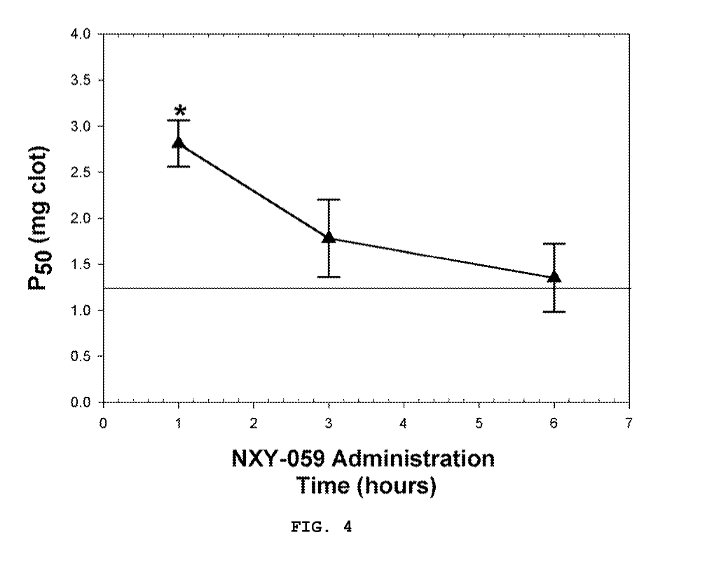

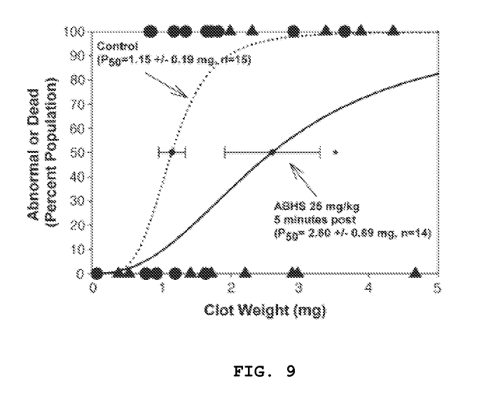

Effect of bolus injections of ABHS on behavior following embolic strokes: Either vehicle or bolus injections of ABHS were administered intravenously over 1 min starting 5 min following small clot embolization. In this series of studies, ABHS was administered at 25 mg/kg based upon the findings of Lapchak (2004). Subsequently, behavioral analysis was conducted 24 h following treatment. Table 1 provides a series of representative raw data used to construct quantal analysis curve showing the effects of ABHS (25 mg/kg) on behavior. As shown in Table 1 and then graphically in FIG. 9, ABHS at 25 mg/kg significantly (P<0.05) improved stroke induced behavioral deficits and increased the P50 value to 2.60±0.69 mg (n=14). FIG. 3 also shows a graphical representation of the raw data presented in Table 1 superimposed on the theoretical quantal analysis curves. For the superimposed graphs, normal animals are plotted on the y-axis at 0, and abnormal animals are plotted at 100. The figure shows that there is positive correlation between the data (circles or triangles) and the statistically fitted quantal curve. The ABHS-induced improvement in behavior is directly correlated with an increase in the number of animals which are behaviorally “normal” as shown on the y-axis plotted at 0 (at doses above 1.5 mg). The P₅₀ value for the control group, which was run in parallel to the ABHS-treated group, was 1.15±0.19 mg (n=15). However, when ABHS (25 mg/kg) was administered 60 or 180 min following embolization, there was no significant behavioral improvement (Table 2, P>0.05). It should be noted that IV ABHS administration to rabbits resulted in sedation (characterized by loss of righting reflex), which lasted 2-3 h after drug injection.

Table 1 shows the effect of ABHS (25 mg/kg) on clinical rating scores following small clot embolization of rabbits: raw quantal analysis data. Behavioral results are expressed as Normal or Abnormal rabbits for each clot dose shown in milligrams (mg). (n) is the number of animals in each group.

TABLE 1

| Control | | | ABHS (5 min) | | |
|---|---|---|---|---|---|
| Clot dose (mg) | Norm | Abnorm | Clot Dose | Norm | Abnorm |
| 0.07 | 1 | 0 | 0.38 | 1 | 0 |
| 0.77 | 1 | 0 | 0.53 | 1 | 0 |
| 0.78 | 1 | 0 | 1.42 | 1 | 0 |
| 0.83 | 0 | 1 | 1.60 | 1 | 0 |
| 0.85 | 1 | 0 | 1.72 | 1 | 0 |
| 0.93 | 1 | 0 | 1.99 | 0 | 1 |
| 1.18 | 0 | 1 | 2.21 | 0 | 1 |
| 1.20 | 1 | 0 | 2.31 | 1 | 0 |
| 1.35 | 0 | 1 | 2.89 | 0 | 1 |
| 1.64 | 1 | 0 | 2.97 | 1 | 0 |
| 1.65 | 0 | 1 | 3.38 | 0 | 1 |
| 1.72 | 0 | 1 | 3.88 | 0 | 1 |
| 1.82 | 0 | 1 | 4.35 | 0 | 1 |
| 2.65 | 0 | 1 | 4.67 | 1 | 0 |
| 2.91 | 0 | 1 | | | |
| P₅₀ = 1.15 ± 0.19 | | | P₅₀ = 2.60 ± | | |
| mg(15) | | | 0.69(14) | | |

Table 2 shows the effect of ABHS injections on clinical rating scores following an embolic stroke: a time-window study. This table depicts the effects of embolism and ABHS treatment (25 mg/kg) on P50 values measured 24 h following embolization. ABHS administered 5 min following embolization significantly increased clinical rating scores compared to vehicle control.

TABLE 2

| Treatment group | P₅₀ (mg) | n | Observation |
|---|---|---|---|
| A. | | | |
| Vehicle control (5 min group) | 1.15 ± 0.19 | 15 | Normal |
| 5 min | 2.60 ± 0.69 | 14 | Sedation |
| B. | | | |
| Vehicle Control (cumulative) | 1.18 ± 0.25 | 24 | Normal |
| 60 min | 1.05 ± 0.24 | 17 | Sedation |
| 180 min | 1.42 ± 0.35 | 17 | Sedation |

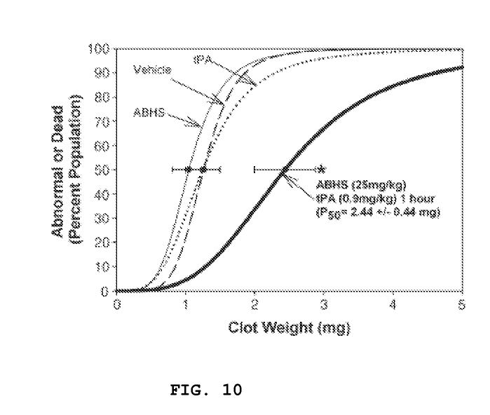

Effect of ABHS in combination with tPA on behavior following embolic strokes: In previous studies, it was determined that drug combination therapy could increase the therapeutic window for thrombolytics such as tPA or TNK. The present studies identified the effects of treating embolized rabbits with ABHS in the absence or presence of tPA. ABHS was given at 25 mg/kg, the dose found to be effective at improving behavioral function in the RSCEM. In the first series of experiments, ABHS given 60 min postembolization, with tPA (3.3 mg/kg) injected starting 60 min after embolization. With tPA alone, a significant increase in the P50 (P₅₀=2.69±0.19 mg, n=15) was noted compared to the control group (P₅₀=1.18±0.25 mg, n=24). When the two drugs were administered in combination starting 60 min after embolization, there was no additional behavioral improvement (P₅₀=2.88±0.26 mg, n=15). The observation that the combination did not significantly increase P₅₀ values compared to monotherapy at the administration time of 60 min when tPA is maximally effective suggests a “ceiling” effect caused by the highly efficacious dose of tPA but does not exclude the possibility of a positive result were different treatment times or a sub-maximal dose of tPA used. This hypothesis was tested in two ways. First, a sub-optimal dose of tPA (0.9 mg/kg), which by itself did not significantly increase the P50 value (1.25±0.25 mg, n=17), was used in combination with ABHS (25 mg/kg) given 60 min post-embolization, also which did not significantly increase the P50 value (1.05±0.24 mg, n=17). A statistically significant increase (P<0.05) in the P₅₀ value (2.44±0.44 mg, n=17) was observed with this combination, suggesting a synergistic effect of the drugs on behavioral improvement (FIG. 10). Second, tPA (3.3 mg/kg) was administered in combination with ABHS (25 mg/kg) starting 180 min following embolization, a treatment time when neither drug alone had an effect on behavioral outcome following embolic strokes. In this group, there was also a significant improvement of behavioral function measured as an increase in P₅₀ to 2.31±0.48 mg (n=17) (FIG. 7).

Currently, the only FDA-approved treatment for acute ischemic stroke (AIS) is the thrombolytic, tissue plasminogen activator (tPA; Alteplase; Activase). It has been proposed that both the spin trap agent, NXY-059 (Cerovive) and Tenecteplase (TNK-tPA), which are currently in Phase III clinical trials, may also be useful for the treatment of ischemic stroke. However, there is little information available concerning the dose-response profiles or therapeutic window for NXY-059 in a validated embolic stroke model, nor is there information available pertaining to the effects of combining NXY-059 with Tenecteplase. Thus, the pharmacological profile of NXY-059 on behavioral outcome following small clot embolic strokes in rabbits when administered alone or in combination with Tenecteplase was determined in the present studies. The therapeutic window for NXY-059 was also determined by administering the drug 1, 3 or 6 hours following embolic strokes. Lastly, in combination studies, NXY-059 was given concomitantly with Tenecteplase 1 or 6 hours following embolization. In the vehicle control group, the P50 value (mg of clots that produce behavioral deficits in 50% of the rabbits) measured 24 hours following embolism was 1.20±0.15 mg, and this was increased by 100-134% if NXY-059 (1-100 mg/kg) was administered following embolization. If NXY-059 was administered beginning 3 or 6 hours following embolization there was no significant behavioral improvement. If NXY-059 (100 mg/kg) and Tenecteplase (0.9 mg/kg) were administered concomitantly 1 hour post-embolization, no additional behavioral improvement was measured compared to either drug alone. However, if the drugs were administered 6 hours following embolization, a statistically significant reduction of behavioral deficits was measured. This study shows that NXY-059 is neuroprotective over a wide range if administered early following an embolic stroke. In addition, the study shows that NXY-059 can be administered in combination with Tenecteplase to provide additional behavioral improvement at extended delays following embolization.

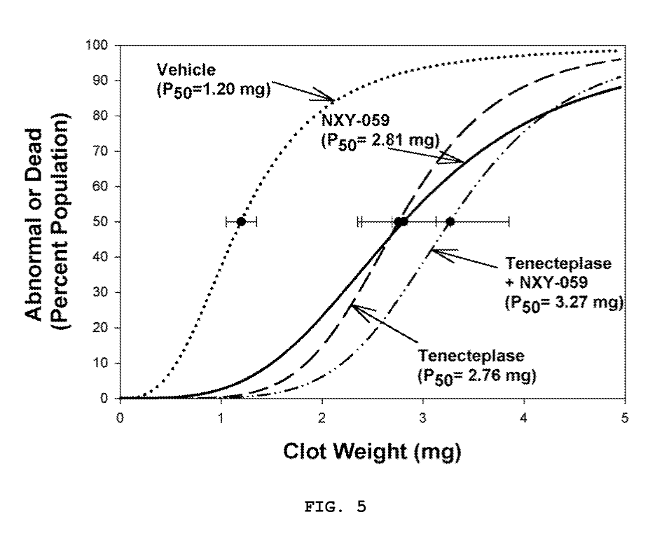

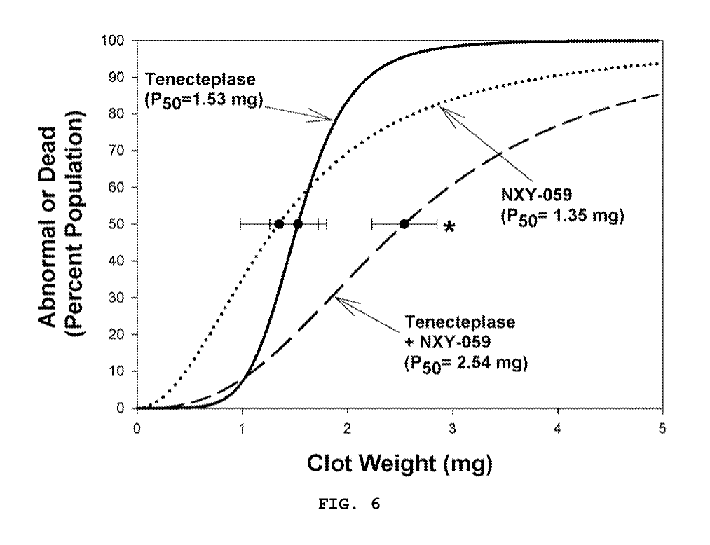

Furthermore, the data (see, FIGS. 5 and 6) demonstrate the pharmacological effects of 3-alpha-ol-5-beta-pregnan-20-one hemisuccinate (ABHS), a neurosteroid that inhibits excitatory amino acid receptor function increases the therapeutic window for thrombolytic agents. The present studies indicate that treatment regimens for combination therapy using, for example, ABHS and tPA reveal additive or synergistic effects in the RSCEM. The present data indicates that the co-administration of ABHS (25 mg/kg) and tPA (3.3 mg/kg) was safe because negative behavioral consequences of the combination therapy were not observed.

In addition, while each exemplary drug can independently improve behavioral performance to a similar extent, in the present study it was found that the concomitant administration of ABHS and tPA 3 h following embolization significantly increased the P₅₀ value (i.e., improved behavioral performance). The P₅₀ value measured for the drug combination group was significantly different from that measured for ABHS when administered 3 h following embolization. This result indicated that the behavioral effect of the drug combination administered at 3 h was similar in magnitude to either ABHS or tPA alone when administered 5 min and 1 h following embolization, respectively.

Moreover, in the present study, it was found that administration of ABHS (25 mg/kg) in combination with low-dose tPA (0.9 mg/kg), doses of each drug that do not result in behavioral improvement following embolic strokes in rabbits when given independently starting 60 min following embolization, had a synergistic effect. Delayed administration of ABHS in combination with a low dose of the tPA presented a superior behavioral improvement profile compared to either drug alone. The synergism between ABHS and tPA indicates that it is possible to administer a lower dose of thrombolytic, thereby reducing complications associated with thrombolytic therapy (e.g., hemorrhage), while still providing maximal behavioral improvement. This is supported by previous studies which showed that a low dose of tPA (0.9 mg/kg) increased hemorrhage rate by only 28%, compared to the high dose (3.3 mg/kg), which increased the rate by 52%. Further, ABHS may reduce reperfusion-induced injury caused by tPA and consequently protect neurons that are at risk. There is now a link between NMDA receptors, in particular NR1/NR2-containing receptors and the deleterious side effects of tPA administration. The observation that ABHS and other neuroactive steroids modulate NMDA receptors composed of NR1/NR2 indicates that the synergistic effect of the combination of ABHS and tPA can be due in part to limiting tPA-induced toxicity.

Polyphenol Administration: In another embodiment, methods and compositions of the invention may include polyphenol, or a derivative thereof, for the treatment of embolic stroke.

Using the RSCEM model as described above male New Zealand white rabbits weighing 2.2-2.6 Kg were anesthetized and a catheter was inserted into the common carotid artery through which microclots were injected. Briefly, the bifurcation of one carotid artery was exposed and the external carotid was ligated just distal to the bifurcation. A catheter was inserted into the common carotid and secured with ligatures. The incision was closed around the catheter so that the distal end was accessible outside the animal's neck. The catheter was then filled with heparinized saline and plugged with an injection cap. Rabbits were allowed to recover from anesthesia until they were awake and behaving normally.

Embolic Strokes were generated by drawing blood from one or more donor rabbits and allowed to clot for 3 h at 37° C. The large blood clots were then suspended in PBS with 0.1% bovine serum albumin (BSA) and Polytron-generated fragments were sequentially passed through metal screens and nylon filters. The resulting small clot suspension was then labeled with ⁵⁷Co containing NEN-Trac microspheres. The addition of microspheres allowed for the calculation of the dose of clots that becomes lodged in the brain following embolization. For embolization, rabbits were placed in Plexi-glass restrainers, the catheter injection cap was removed and the heparinized saline cleared from the carotid catheter system. Then 1 ml of a clot particle suspension containing small sized blood clots and ⁵⁷Co NEN-Trac microspheres was injected through the catheter into the brain, which was then flushed with 5 ml of normal sterile saline. Rabbits are fully awake during the embolization procedure and they are self-maintaining (i.e.: they do not require artificial respiration or other external support). This allows for immediate observation of the effects of embolization on behavior at the time of clot injection and thereafter.

To evaluate the quantitative relationship between clot dose and behavioral deficits, logistic S shaped quantal analysis curves are fitted to the dose response data as described herein. A wide range of clot doses was used resulting in behaviorally normal and abnormal animals. In the absence of a neuroprotective treatment regimen, small numbers of microclots cause no grossly apparent neurologic dysfunction and large numbers of microclots invariably caused encephalopathy or death. Using a simple dichotomous rating system, with a reproducible composite result and low inter-rater variability (<5%), each animal was rated by a naïve-observer as either behaviorally normal or abnormal. Abnormal rabbits include those with one or more of the following symptoms: ataxia, leaning, circling, lethargy, nystagmus, loss of balance, loss of limb/facial sensation and occasionally, paraplegia. A separate curve is generated for each treatment condition and a statistically significant increase in the P₅₀ value or the amount of microclots that produce neurologic dysfunction in 50% of a group of animals compared to control is indicative of a behavioral improvement.

The behavioral data are presented as P₅₀ (Mean±SEM) in mg clots for the number of rabbits in each group (n). P₅₀ values were analyzed using the t-test, which included the Bonferroni correction where appropriate. Drug-treated groups were directly compared to respective control groups from each study. For all experiments in the study, rabbits were randomly allocated into treatment groups before the embolization procedure, with concealment of the randomization guaranteed by using an independent third party. The randomization sequence was not revealed until all post-mortem analyses were complete.

Drugs were administered as follows: (1) CGA (50 mg/kg) purchased from Cayman Chemicals Co. was dissolved in 50% hydroxypropylcyclodextrin (Cerestar Inc, Hammond, Ind.) in 0.9% saline for intravenous injection. CGA was given intravenously starting 5 minutes, 1 hour or 3 hours post-embolization; (2) tPA (3.3 mg/kg tPA) was given 1 hour or 3 hours post-embolization, with 20% of the dose as a bolus injection over one minute, followed by the remainder infused over 30 min. Genentech, Inc. (South San Francisco, Calif.) supplied tPA lyophilized in 50 mg configurations, containing 50 mg tPA (29 million IU), 1.7 mg L-arginine, 0.5 g phosphoric acid and less than 4 mg Polysorbate 80, the same formulation used clinically, that was then reconstituted with sterile water (1 mg/ml); (3) For combination studies, CGA (50 mg/kg) and tPA (3.3 mg/kg) were administered beginning 3 hours post-embolization.

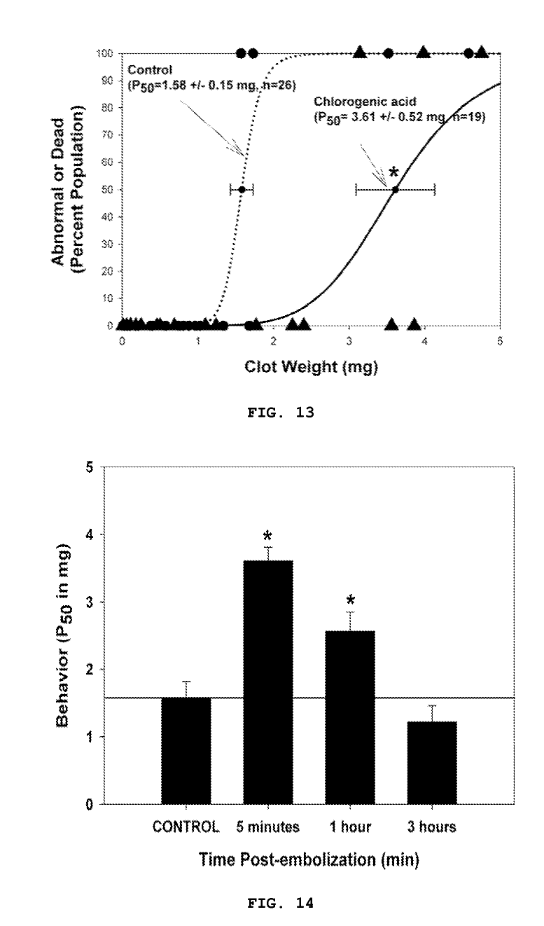

In a first series of experiments, either vehicle or bolus injections of CGA was administered over 1 minute starting 5 minutes following embolization. In a second series of studies, CGA was administered at 50 mg/kg. Behavioral analysis was conducted 24 hours following treatment, which allowed for the construction of quantal analysis curves. FIG. 13 shows a graphical representation of the raw data that is superimposed on the quantal analysis curves. For the superimposed graphs, normal animals are plotted on the y-axis at 0 and abnormal animals are plotted at 100. The figure shows that there is positive correlation between the data (circles or triangles) and the statistically fitted quantal curve. CGA significantly (p<0.05) reduced stroke-induced behavioral deficits and increased the P₅₀ value to 3.61±0.52 mg. The CGA-induced improvement in behavior is directly correlated with an increase in the number of animals which are behaviorally “normal” as shown on the y-axis plotted at 0. The P₅₀ value for the vehicle-treated control group was 1.58±0.15 mg (n=26). CGA was shown to be safe when administered to embolized rabbits at the pharmacological dose described above. That is, there were no apparent side-effects observed upon IV administration of the drug.

Because a time interval of 5 minutes following a stroke is not practical in many clinical situation where patients normally present 3-12 hours following a stroke a set of experiments analyzed the therapeutic window for CGA. For this, rabbits were embolized and were then treated with CGA either 1 or 3 hours following the stroke. FIG. 14 shows the results of the therapeutic window studies. When CGA was administered as a single bolus injection at 1 hour post-embolization, there was a significant increase in the P₅₀ value to 2.57±0.28 mg (n=18) compared to the baseline cumulative control P₅₀ value 1.58±0.15 mg (n=26). However, CGA was not effective at improving clinical rating scores when given 3 hours following embolization (P50=1.22±0.24 mg, n=18).

In another series of studies, the effect of CGA (50 mg/kg) administered in combination with tPA (3.3 mg/kg) was analyzed (e.g., negative, positive (additive or synergistic) or neutral (no effect)) on behavioral outcome. These experiments assessed the effects of treating embolized rabbits 3 hours following embolization with CGA in the absence or presence of tPA. The 3 hour post-embolization time was chosen for the study because it is a time when neither CGA nor tPA are effective at improving clinical rating scores when administered as a monotherapy. In addition, in previous studies a 3-hour delay is useful to reveal synergistic effects of drug treatment, especially in combination with tPA or tenecteplase.

For combination studies with the thrombolytic tPA, tPA at a standard dose of 3.3 mg/kg, a dose that reduces stroke-induced behavioral deficits when given 1 hour following embolization was used. In this study, tPA significantly increased the P₅₀ to 2.89±0.29 mg (n=17) when administered 1 hour after embolization, but not 3 hours after embolization (P₅₀=1.54±0.27 mg, n=18). However, when tPA (3.3 mg/kg) was administered concomitantly with CGA (50 mg/kg) starting 3 hours following embolization, there was a significant increase in behavioral function and a reduction of stroke-induced motor deficits as evidenced by an increase in the P₅₀ value to 3.40±0.76 mg, n=23). The combination of drugs as also shown to be safe in rabbits as it improved behavior.

The literature suggests that NXY-059 would have limited pharmacological effects and efficacy within cells because the molecule is hydrophilic (lipophobic) and does not readily cross the BBB. The absence of BBB penetration is considered to be a severe limitation for a drug and it is hypothesized to be one of the primary reasons why NXY-059 was not efficacious in advanced clinical trials across a broad population of stroke patients. With respect to BBB penetration, it is important to point out that CGA is a more hydrophobic compound than NXY-059. The hydrophobicity of CGA has been described as intermediate between vitamin C, which is highly hydrophilic and vitamin E, which is highly hydrophobic thus CGA has a greater ability to cross the BBB so that it effectively blocks the deleterious effects of free radicals within both vascular and cellular compartments. Thus, CGA itself or even more hydrophobic CGA analogs that could be synthesized are be useful to treat stroke patients.

When CGA (50 mg/kg) and an optimal dose of tPA (3.3 mg/kg) were administered to embolized rabbits at 3 hours, a time when neither drug alone can improve stroke-induced behavioral deficits, the data demonstrate there was a significant extension of the therapeutic window for each of the drugs. Moreover, the maximal increase in P₅₀ was similar to that of each drug when tested independently. The observation that the combination is effective when given 3 hours following embolization in rabbits demonstrates that this combination treatment is useful in a clinical situation up to 9 hours following a stroke. Furthermore, the administration of the combination of CGA and tPA was safe, no negative behavioral consequences of administration of the combination of drugs were observed. The synergism between CGA and tPA is similar to that of previous combination studies in the RSCEM that used various NMDA antagonists and/or antioxidants.

The neuroprotective effects of CGA may be partially attributed to the free radical scavenging abilities of the molecule. It is also interesting to note that CGA has recently been described as a strong metalloproteinase-9 (MMP-9) inhibitor. The suppression of MMP activity may be an important component of CGA's mechanism of action and may contribute to neuroprotection and improved clinical scores following embolic strokes. Since all three mechanisms, free radicals, inflammation and MMP activity are important in the progression of damage resulting from an ischemic stroke, it is possible that one or more of the mechanisms may be involved in CGA-induced neuroprotection and behavioral improvements. The exact mechanism(s) involved in the neuroprotection and behavioral improvements resulting from CGA treatment in embolized rabbits remain to be elucidated and are currently being studied. If a linear correlation exists between dosing in rabbits and humans, then the dose required by a 70 kg human would be approximately 3500 mg.

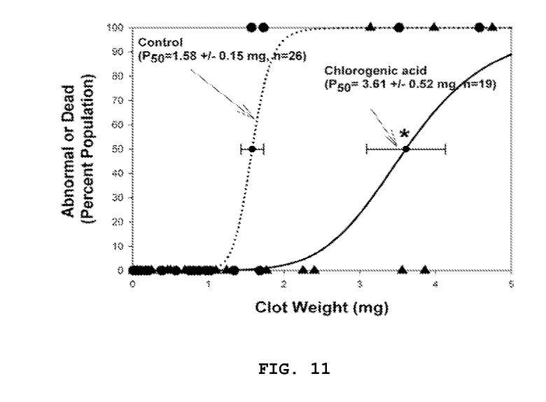

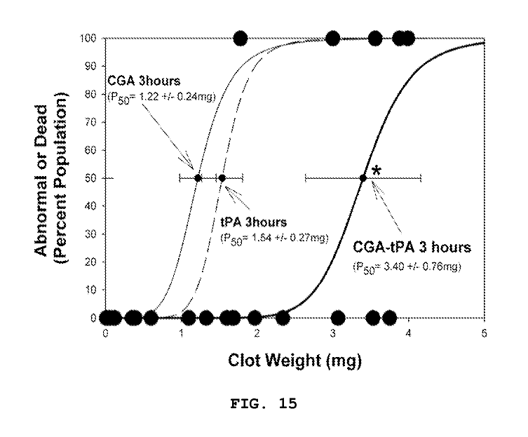

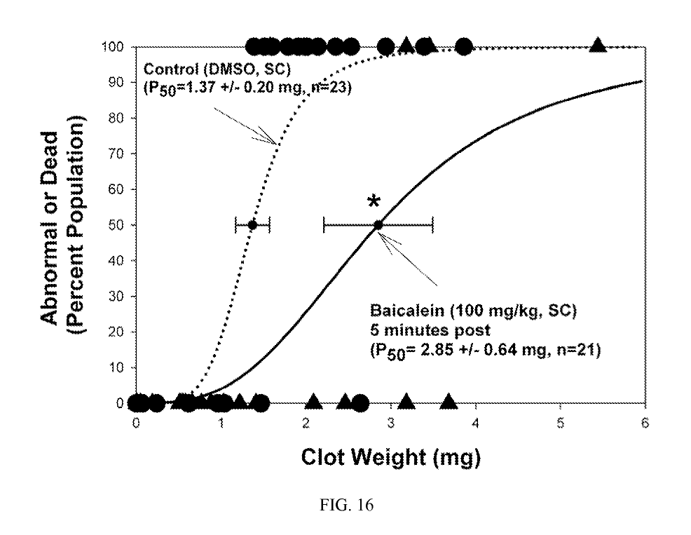

As shown in FIGS. 1, 11-16 behavioral improvements are obtainable following treatment with a polyphenol, such as chlorogenic acid. FIGS. 1, 11-16 show behavioral improvements following chlorogenic acid treatment alone or in combination with a thrombolytic agent. Rabbits were embolized with small blood clots as described previously. Following the stroke, rabbits were treated with chlorogenic acid or a combination therapy via the marginal ear vein. The control curve (dotted line) has a P₅₀ value of 1.58±0.15 mg (n=26). Chlorogenic acid treatment (50 mg/kg IV) initiated 5 minutes following embolization increased the P₅₀ value to 3.61±0.52 mg (n=19, *P<0.05) (dark solid line) (see, e.g., FIG. 1). The dark circles ● represent the raw data from the control group and the triangles ▴ represent the raw data for the chlorogenic acid-treated group. A normal animal for a specific clot weight is represented by a ● symbol plotted at 0 on the y-axis, whereas an abnormal animal for a specific clot weight is represented by a ▴ symbol plotted at 100 on the y-axis.

Chlorogenic acid administration resulted in significant behavioral improvement following embolic strokes when measured 24 hours following embolization or blood clot injection indicating that the administration of an antioxidant such as polyphenol, or a derivative thereof, may be used to treat stroke.

The data demonstrate that CGA may be useful as both a monotherapy and in combination with thrombolytics for the treatment of AIS. The short therapeutic window for CGA following an embolic stroke suggests that patients presenting within a few hours of a stroke may ultimately receive the clinical benefits of CGA monotherapy. However, the results with CGA in combination with tPA are promising because the two drugs with rather different modes of action act synergistically to extend the therapeutic window for intervention following an embolic stroke.

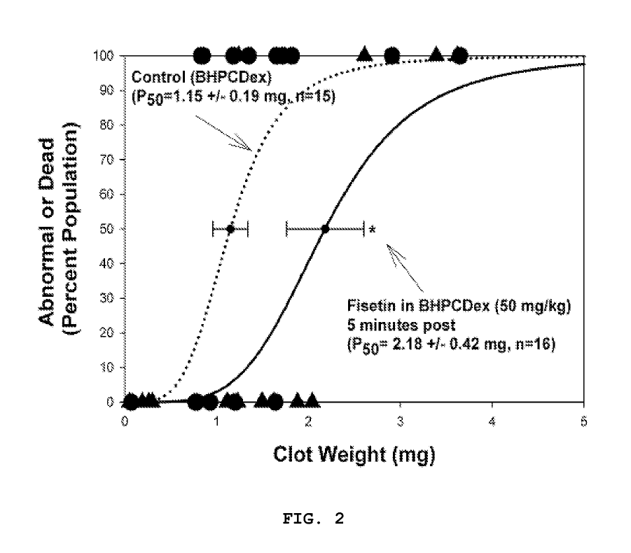

Additional polyphenols demonstrated similar improvements in behavior following the rabbit stroke model described above. For example, FIG. 2 shows behavioral improvements following fisetin treatment. Rabbits were embolized with small blood clots according to methods described previously. Following the stroke, rabbits were treated with fisetin via the marginal ear vein. The control curve (dotted line) has a P₅₀ value of 1.15±0.19 mg (n=15). Fisetin treatment (50 mg/kg IV) initiated 5 minutes following embolization increased the P₅₀ value to 2.18±0.42 mg (n=16, *P<0.05) (dark solid line). The dark circles ● represent the raw data from the control group and the triangles ▴ represent the raw data for the fisetin-treated group. A normal animal for a specific clot weight is represented by a ● symbol plotted at 0 on the y-axis, whereas an abnormal animal for a specific clot weight is represented by ▴ A symbol plotted at 100 on the y-axis. FIG. 16 shows that the natural product baicalein, when administered after an embolic stroke, can significantly reduce stroke-induced behavioral deficits.

The examples set forth above are provided to give those of ordinary skill in the art a complete disclosure and description of how to make and use the embodiments of the methods, treatments and compositions of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety.

1. A method of treating stroke comprising:
contacting a subject suffering from a stroke with:
a) an NMDA receptor antagonist;
b) an agent that increases reperfusion of an affected area in an amount sufficient to allow for the penetration of a spin trap agent; and
c) a spin trap agent in an amount sufficient to reduce cell and/or tissue damage.

2. The method of claim 1, wherein the agent is a thrombolytic agent.

3. The method of claim 2, wherein the thrombolytic agent is selected from the group consisting of alteplase, tenecteplase, reteplase, streptase, abbokinase, pamiteplase, nateplase, desmoteplase, duteplase, monteplase, reteplase, lanoteplase, Prolyse™, microplasmin, Bat-tPA, BB-10153, and any combination thereof.

4. The method of claim 2, wherein the thrombolytic agent is tenectaplase.

5. The method of claim 1, wherein the spin trap agent is selected from the group consisting of nitrone and nitroso spin trap compounds.

6. The method of claim 5, wherein the nitrone and nitroso spin trap compound is selected from the group consisting of disodium 2,4-disulfophenyl-N-tert-butylnitrone (NXY-059), N-t-butyl-a-phenylnitrone, 3,5-dibromo-4-nitrosobenzenesulfonic acid, 5,5-dimethyl-1-pyrroline N-oxide, 2-methyl-2-nitrosopropane, nitrosodisulfonic acid, a-(4-pyridyl-1-oxide)-N-t-butylnitrone, 3,3,5,5-tetramethylpyrroline N-oxide, 2,4,6 tri-t-butylnitrosobenzene, PTIYO (4-phenyl-2,2,5,5-tetramethyl imidazolin-1-yloxy-5-oxide), tempol (4-hydroxy 2,2,6,6-tetramethylpiperidine-1-oxyl), and any combination thereof.

7. The method of claim 1, wherein the spin trap agent is NXY-059.

8. The method of claim 1, wherein the agent is tenectaplase and the spin trap agent is NXY-059.

9. The method of claim 1, wherein the subject is contacted with the NMDA receptor antagonist prior to contact with the agent and the spin trap agent.

10. The method of claim 1, wherein the subject is contacted simultaneously with the agent and the spin trap agent.

11. The method of claim 1, wherein the subject is contacted with the agent prior to contacting the subject with spin trap agent.

12. The method of claim 1, wherein the NMDA receptor antagonist is selected from the group consisting of 3-alpha-ol-5-beta-pregnan-20-one hemisuccinate (ABHS), ketamine, memantine, dextromethorphan, dextrorphan, and dextromethorphan hydrobromide.

13. A method of treating stroke comprising contacting a subject suffering from a stroke with an agent that increases reperfusion of an affected area; and contacting the subject with an NMDA receptor antagonist in an amount sufficient to reduce cell and/or tissue damage.

14. The method of claim 13, wherein the agent is a thrombolytic agent.

15. The method of claim 13, wherein the thrombolytic agent is selected from the group consisting of alteplase, tenecteplase, reteplase, streptase, abbokinase, pamiteplase, nateplase, desmoteplase, duteplase, monteplase, reteplase, lanoteplase, Prolyse™, microplasmin, Bat-tPA, BB-10153, and any combination thereof.

16. The method of claim 13, wherein the NMDA receptor antagonist is selected from the group consisting of 3-alpha-ol-5-beta-pregnan-20-one hemisuccinate (ABHS), ketamine, memantine, dextromethorphan, dextrorphan, and dextromethorphan hydrobromide.

17. The method of claim 14, wherein the agent is a thrombolytic agent and the NMDA receptor antagonist is either ABHS or memantine.

18. The method of claim 17, wherein the thrombolytic agent is tPA or tNKA.

19. A formulation comprising a thrombolytic agent and (i) an NMDA receptor antagonist, or (ii) a combination of an NMDA receptor antagonist and a spin trap agent.

20. The formulation of claim 19, wherein the thrombolytic is selected from the group consisting of alteplase, tenecteplase, reteplase, streptase, abbokinase, pamiteplase, nateplase, desmoteplase, duteplase, monteplase, reteplase, lanoteplase, Prolyse™, microplasmin, Bat-tPA, BB-10153, and any combination thereof.

21. The formulation of claim 19, wherein the spin trap agent is selected from the group consisting of disodium 2,4-disulfophenyl-N-tert-butylnitrone (NXY-059), N-t-butyl-a-phenylnitrone, 3,5-dibromo-4-nitrosobenzenesulfonic acid, 5,5-dimethyl-1-pyrroline N-oxide, 2-methyl-2-nitrosopropane, nitrosodisulfonic acid, a-(4-pyridyl-1-oxide)-N-t-butylnitrone, 3,3,5,5-tetramethylpyrroline N-oxide, 2,4,6-tri-t-butylnitrosobenzene, PTIYO (4-phenyl-2,2,5,5-tetramethyl imidazolin-1-yloxy-5-oxide), tempol (4-hydroxy 2,2,6,6-tetramethylpiperidine-1-oxyl), and any combination thereof.

22. The formulation of claim 19, wherein the thrombolytic agent is tenectaplase and the spin trap agent is NXY-059.

23. The formulation of claim 19, wherein the NMDA receptor antagonist is selected from the group consisting of 3-alpha-ol-5-beta-pregnan-20-one hemisuccinate (ABHS), ketamine, memantine, dextromethorphan, dextrorphan, and dextromethorphan hydrobromide.

24. The formulation of claim 19, wherein the thrombolytic agent is tPA and the NMDA receptor antagonist is ABHS.

25. The formulation of claim 19, wherein the thrombolytic agent is tPA and the NMDA receptor antagonist is memantine.

26. The formulation of claim 19, wherein the thrombolytic agent is tNKA and the NMDA receptor antagonist is ABHS.

27. The formulation of claim 19, wherein the thrombolytic agent is tNKA and the NMDA receptor antagonist is memantine.
